(19) United States
(12) Patent Application Publication
(10) Pub. No.: US 2015/0190078 A1
(43) Pub. Date: Jul. 9, 2015

(54) POWER EFFICIENT PULSE OXIMETRY SYSTEM

(71) Applicant: COVIDIEN LP, MANSFIELD, MA (US)

(72) Inventor: Daniel Lisogurski, Boulder, CO (US)

(21) Appl. No.: 14/587,483

(22) Filed: Dec. 31, 2014

Related U.S. Application Data
(60) Provisional application No. 61/925,431, filed on Jan. 9, 2014.

(51) Int. Cl. A61B 5/1455; A61B 5/00; A61B 5/024

(52) U.S. Cl. CPC A61B 5/14551; A61B 5/02416; A61B 5/7225

(57) ABSTRACT

Various systems and methods for patient monitoring are provided. A monitor may include a sensor interface configured to obtain a signal from a sensor applied to a patient. The monitor may also include data processing circuitry configured to obtain a heart rate of the patient, select a filter based at least in part on the heart rate, and filter the signal using the selected filter to reduce noise at frequencies other than the heart rate. Additionally, the monitor may include a light source drive circuitry configured to reduce power to a light source of the sensor after the selected filter is applied to the signal.

### CROSS REFERENCE TO RELATED APPLICATIONS

This application claims the benefit of U.S. Provisional Patent Application No. 61/925,431, filed Jan. 9, 2014, entitled “Power Efficient Pulse Oximetry System,” which is incorporated by reference herein in its entirety.

### BACKGROUND

The present disclosure relates generally to medical devices and, more particularly, to power efficient patient monitoring devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring certain physiological characteristics of a patient. Such devices provide doctors and other healthcare personnel with the information they need to provide the best possible healthcare for their patients. As a result, such monitoring devices have become an indispensable part of modern medicine. For example, photoplethysmography is a common technique for monitoring physiological characteristics of a patient, and one device based upon photoplethysmography techniques is commonly referred to as pulse oximetry. Pulse oximeters may be used to measure and monitor various blood flow characteristics of a patient. A pulse oximeter may be utilized to monitor the blood oxygen saturation of hemoglobin in arterial blood, the volume of individualized blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

A patient in a hospital setting may be monitored by a pulse oximetry sensor that includes a light source. The light source may be a light emitting diode that emits light into the patient's tissue, and the light is subsequently detected by a photodetector that generates a signal indicative of a physiological parameter of the patient (e.g., blood oxygen saturation). The signal generated by the photodetector typically includes noise components introduced by ambient light, patient movement, or the like. To maintain sufficiently high signal-to-noise ratios (SNR) for patient monitoring, the light source is typically driven with a large amount of current to generate more light for emission into the patient's tissue and for detection by the photodetector. However, the large drive current causes the light source to consume a large amount of power.

### BRIEF DESCRIPTION OF THE DRAWINGS

Advantages of the disclosed techniques may become apparent upon reading the following detailed description and upon reference to the drawings in which:

FIG. 1 is a block diagram of a medical monitoring system, in accordance with an embodiment;

FIG. 2 is a block diagram of a medical monitoring system with reduced power consumption, in accordance with an embodiment;

FIG. 3 is a block diagram of another medical monitoring system with reduced power consumption, in accordance with an embodiment;

FIG. 4 is a process flow diagram of a method of operating a monitoring system with reduced power consumption, in accordance with an embodiment;

FIG. 5 is a process flow diagram of a method of adjusting a filter for operating a monitoring system with reduced power consumption, in accordance with an embodiment; and

FIG. 6 is an example of a plethysmography signal having a baseline that oscillates in response to a respiration rate of a patient.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure is generally directed toward power efficient medical monitoring systems and methods. Embodiments disclosed herein relate to pulse oximeters to facilitate discussion. However, it should be understood that the techniques disclosed herein may be applied to and/or adapted for use with any of a variety of patient monitors and associated patient sensors. Pulse oximeters typically include a sensor having one or more light sources (e.g., light emitting diodes or LEDs) that emit light into a patient's tissue and one or more detectors (e.g., photodetectors) that detect the light after the light passes through the patient's tissue. The detector may generate a signal indicative of a physiological parameter of the patient, such as blood oxygen saturation. However, such signals typically have noise components due to ambient light or patient movement, for example. To minimize the effects of such noise, typically pulse oximetry systems drive the light source with a large amount of current. While the high current drive may minimize the effects of noise and provide a signal having high SNR for patient monitoring, the high current drive also consumes a significant amount of power. Thus, the present disclosure is directed to techniques for operating patient monitoring systems, such as pulse oximetry systems, in a power efficient manner. For example, certain techniques described herein may determine and/or select suitable filters based at least in part on the patient's heart rate and/or respiration rate. Such filters may be applied to the signal generated by the detector to reduce noise acquired at a front end, which in turn enables the system to adjust certain features of the patient monitoring system to reduce power consumed by the system, while maintaining adequate SNR for patient monitoring. For example, the power consumed by the system may be reduced by lowering the current drive to the light source, reducing the duty cycle, adjusting a sampling rate of an analog-to-digital converter (ADC) of the system, or any suitable change to the system.

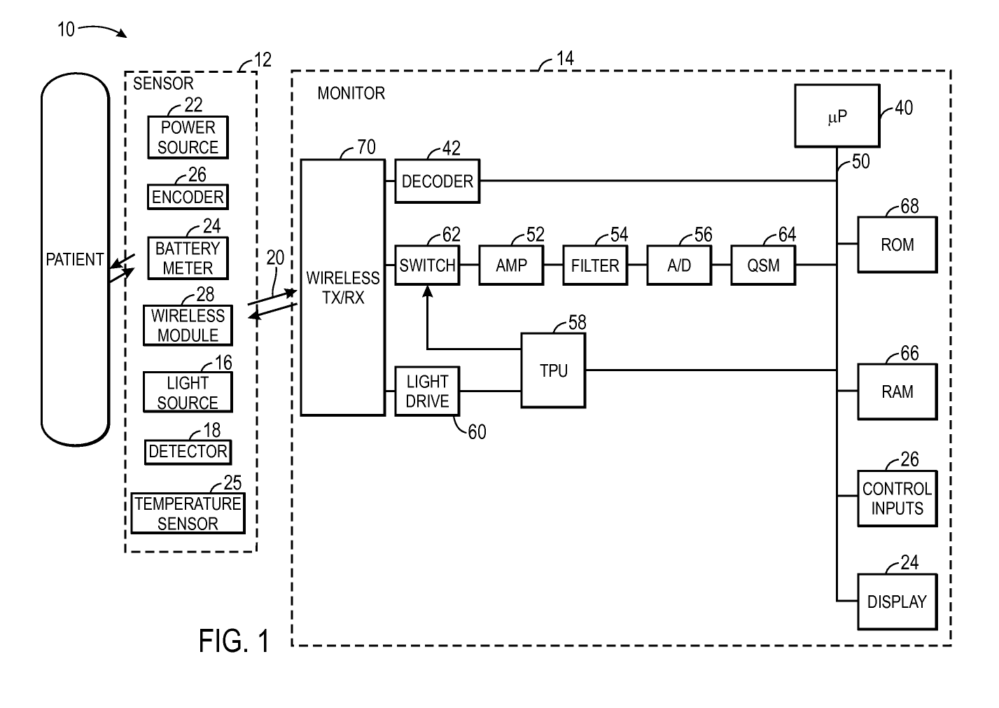

With the foregoing in mind, FIG. 1 depicts a block diagram of one embodiment of a medical monitoring system. As shown, the medical monitoring system is a pulse oximetry system 10 having a sensor 12 and a monitor 14. The sensor 12 may include one or more light sources 16 (e.g., emitter) configured to emit light into a patient's tissue. The sensor 12 may also include one or more detectors 18 configured to detect light from the light source 16 after the light passes through the patient's tissue. The detector 18 may be configured to generate a signal, such as a photoplethysmography signal, based on the detected light. The detector 18 may transmit and/or provide the signal to the monitor 14.

The one or more light sources 16 may be a light emitting diode (LED), a superluminescent light emitting diode, a laser diode or a vertical cavity surface emitting laser (VCSEL). Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. For example, the light from the light source 16 may be used to measure blood pressure, blood oxygen saturation, water fractions, hematocrit, or other physiological parameters of the patient. In certain embodiments, the light source 16 may emit at least two (e.g., red and infrared) wavelengths of light. The red wavelength may be between about 600 nanometers (nm) and about 700 nm, and the IR wavelength may be between about 800 nm and about 1000 nm. However, any appropriate wavelength (e.g., green, yellow, etc.) and/or any number of wavelengths (e.g., three or more) may be used. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In some embodiments, the sensor 12 may be coupled to the monitor 14 via one or more cables. In the embodiment depicted in FIG. 1, the sensor 12 is configured to operate and/or to communicate with the monitor 14 wirelessly 20, without the use of any cables or cords. In some such embodiments, the sensor 12 may include or may be coupled to a power source 22 (e.g., a battery) to supply the sensor 12 with power. By way of example, the battery 22 may be a rechargeable battery (e.g., a lithium ion, lithium polymer, nickel-metal hydride, or nickel-cadmium battery) or may be a single-use battery such as an alkaline or lithium battery. A battery meter 24 may be included in the sensor 12 to provide the expected remaining power of the battery 22 to the monitor 14, for example. In certain embodiments, the sensor 12 may include or may be coupled to one or more additional sensing components, such as a temperature sensor 25, as discussed in more detail below.

The sensor 12 may also include an encoder 26, which may contain information about the sensor 12, such as what type of sensor it is (e.g., a type of sensor, a location where the sensor is to be placed, etc.) and how the sensor 12 is to be driven (e.g., wavelength of light emitted by the light source 16). This information may enable the monitor 14 to select appropriate algorithms and/or calibration coefficients or to derive a filter for estimating the patient's physiological characteristics. The encoder 26 may, for instance, be a memory on which information may be stored for communication to the monitor 14. The encoder 26 may store information related to the wavelength of the light source 16. The encoder 26 may, for instance, be a coded resistor, EEPROM or other coding devices (such as a capacitor, inductor, PROM, RFID, parallel resident currents, or a colorimetric indicator) that may provide a signal to a microprocessor 40 or other processing circuitry of the monitor 14 related to the characteristics of the sensor 12 to enable the microprocessor 40 to determine the appropriate calibration characteristics. In some embodiments, the data or signal from the encoder 26 may be decoded by a detector/decoder 42 in the monitor 14. In some embodiments, the encoder 26 and/or the decoder 42 may not be present.

The microprocessor 40 of the monitor 14 may be coupled to an internal bus 50. The received signal from the sensor 12 may be passed through an amplifier 52, a filter 54, and an analog-to-digital converter 56 (ADC). As discussed in more detail below, the filter 54 may be determined and/or selected by the monitor 14 based at least in part on a time varying waveform characteristic of the signal generated by the detector 18, such as the patient's heart rate and/or respiration rate. Thus, in certain embodiments, the filter 54 may be configured to attenuate noise at frequencies other than the patient's heart rate and/or respiration rate, which in turn may enable the system 10 to reduce the current available to the light source 16 while maintaining adequate SNR for patient monitoring. In certain embodiments, the filter 54 may be accessed and/or selected from a filter bank, which may include multiple filters stored within the monitor 14, the sensor 12, and/or an external device or network.

A time processing unit (TPU) 58 may provide timing control signals to light drive circuitry 60, which may be configured to control and/or to adjust the power consumed by the light source 16. For example, the light drive circuitry 60 may control and/or adjust when the light source 16 of the sensor 12 is activated, and, if multiple light sources are used, the multiplexed timing for the different light sources 16. In certain embodiments, the light drive circuitry 60 may be configured to control a duty cycle and/or to control the maximum current provided to the light source 16 of the sensor 12. Various techniques for controlling and/or adjusting the power consumed by the light source 16 and/or the system 10 are discussed in more detail below.

The TPU 58 may also control the gating-in of signals from sensor 12 through a switching circuit 62. These signals are sampled at the proper time, depending at least in part upon which of multiple light sources is activated, if multiple light sources are used. The digital data may then be stored in a queued serial module (QSM) 64, for later downloading to RAM 66 or ROM 68 as QSM 64 fills up. In addition, the monitor 14 may include a display 24 configured to display information regarding the physiological parameters, information about the system, and/or alarm indications, for example. The monitor 14 may also include various control inputs 26, such as knobs, switches, keys and keypads, touchscreens, buttons, etc., to provide for operation and configuration of the monitor 14.

As noted above, in some embodiments, the sensor 12 and the monitor 14 may communicate wirelessly 20. Thus, the sensor 12 may include a wireless module 70 (e.g., wireless transceiver), and the monitor 14 may include a wireless module 72. The wireless module 70 of the sensor 12 may establish the wireless communication 20 with the wireless module 72 of the monitor 14 using any suitable protocol. By way of example, the wireless modules 70, 72 may be capable of communicating using the IEEE 802.15.4 standard, and may communicate, for example, using ZigBee, WirelessHART, or MiWi protocols. Additionally or alternatively, the wireless modules 70, 72 may be capable of communicating using the Bluetooth standard or one or more of the IEEE 802.11 standards. In an embodiment, the wireless module 70 may include a transmitter (such as an antenna) for transmitting wireless data, and the wireless module 72 includes a receiver (such as an antenna) for receiving wireless data. In an embodiment, the wireless module 70 also includes a receiver for receiving instructions (such as instructions to switch modes), and the wireless module 72 also includes a transmitter for sending instructions to the sensor 12.

Additionally, based at least in part upon the received signals corresponding to the light received by optical components of the sensor 12, the microprocessor 40 may be configured to determine an oxygen saturation, a heart rate, a respiration rate, and/or other physiological parameters using various algorithms. The algorithms may employ certain coefficients, which may be empirically determined and may correspond to the wavelengths of light used. The algorithms and coefficients may be stored in the ROM 68 or other suitable computer-readable storage medium or memory circuitry and accessed and operated according to microprocessor 40 instructions.

Furthermore, one or more functions of the monitor 14 may also be implemented directly in the sensor 12. For example, in some embodiments, the sensor 12 may include one or more processing components configured to calculate the oxygen saturation, the heart rate, the respiration rate, and/or various physiological parameters from the signals obtained from the patient. The sensor 12 may have varying levels of processing power, and may wirelessly output data in various stages to the monitor 14. For example, in some embodiments, the data output to the monitor 14 may be analog signals, such as detected light signals (e.g., pulse oximetry signals or regional saturation signals), or processed data.

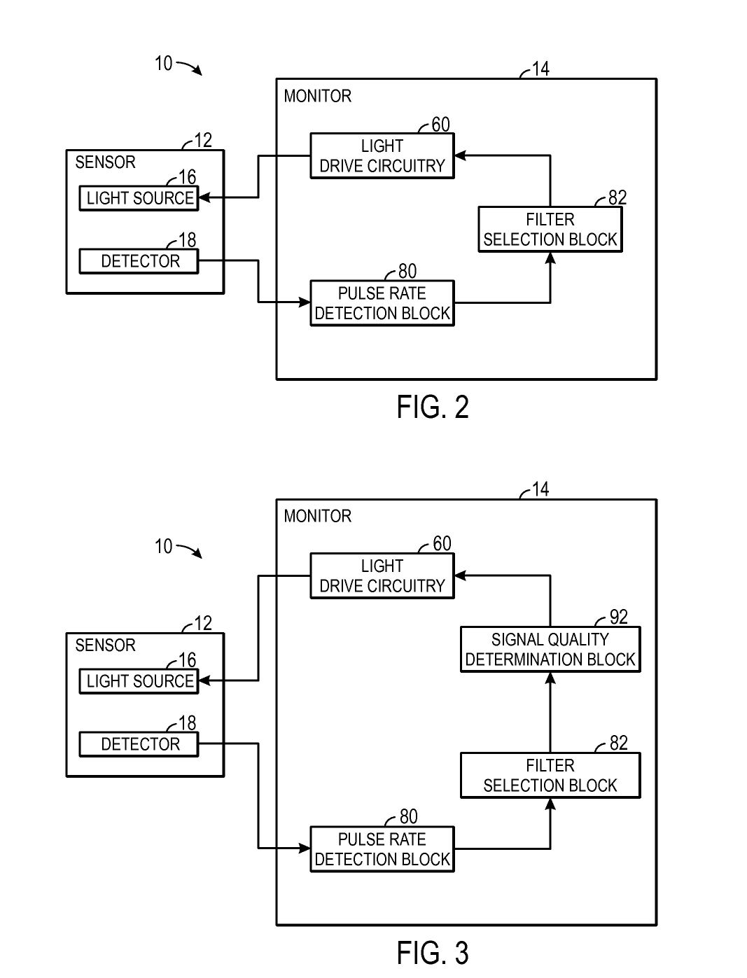

As noted above, it may be desirable for the system 10 to reduce the power consumed by the light source 16 of the sensor 12. Accordingly, FIG. 2 is a block diagram of a medical monitoring system 10 with reduced power consumption, in accordance with an embodiment. Although the embodiment described with respect to FIG. 2 relates to detecting a pulse rate and selecting a filter based on the pulse rate, it should be understood that the system 10 may additionally or alternatively be configured to determine a respiration rate and to select the filter based on the respiration rate (or select one or more filters based on the heart rate and the respiration rate), as described in more detail below. As shown, the system 10 includes a pulse rate detection block 80 (e.g., heart rate detection block), a filter selection block 82, and the light drive circuitry 60. The pulse rate detection block 80 may be data processing circuitry within the monitor 14 and may be generally configured to determine the patient's heart rate. The pulse rate detection block 80 may determine and/or obtain the patient's heart rate in any suitable manner. For example, in some embodiments, the pulse rate detection block 80 may determine the patient's heart rate based on a signal received from the sensor 12. In such embodiments, it may be desirable to determine the patient's heart rate when the light source 16 is driven at high power (e.g., provided with a high maximum current and/or duty cycle) so that the resulting signal generated by the detector 18 has a high SNR that enables the pulse rate detection block 80 to reliably determine the patient's heart rate. In certain embodiments, the pulse rate detection block may receive a user input of the patient's pulse rate. In some embodiments, an external device may be utilized to detect the patient's heart rate. For example, the monitor 14 and/or the pulse rate detection block 80 may receive pulse rate data from an ECG sensor or monitor. In some such cases, detections of a QRS complex from the ECG monitor may be provided to the monitor 14 and/or the pulse rate detection block 80 using a wireless or wired communication protocol.

With the foregoing in mind, at certain times during a monitoring session, the system 10 may operate at a default power level (e.g., default power mode, high power level, high power mode). During operation at the default power level, the light source 16 may be driven with sufficient power to enable the detector 18 to detect the light and to generate a signal having a relatively high SNR (e.g., a signal having a SNR that is at least high enough for reliably determining the patient's heart rate). In some embodiments, the default power level provides a maximum available current of about 40 to 60 mA, about 45 to 55 mA, or about 50 mA to the light source 16. The drive current provided in the default power mode may be based on a preset value or range of values (e.g., a predetermined current that is set at the manufacturing stage or prior to the monitoring session). In certain embodiments, the drive current provided in the default power mode may be based on a target SNR or SNR range (e.g., a predetermined desired SNR range or threshold set at the manufacturing state or prior to the monitoring session), and the drive current may be adaptively controlled to achieve the target SNR.

In certain embodiments, the drive current provided in the default power mode may additionally or alternatively be based on a target temperature or temperature threshold. Thus, the drive current may be selected or limited in order to minimize patient discomfort and/or to minimize the effects of high temperatures at the light source 16. For example, light source 16 temperatures less than or equal to about approximately 41 degrees Celsius or less may typically be maintained for extended periods of time. However, it is generally desirable that temperatures of approximately 42 degrees Celsius be maintained for less than or equal to about eight hours, while temperatures of approximately 43 degrees Celsius be maintained for less than or equal to about four hours. In some cases, the threshold temperature may be approximately 44, 45, 46, 47, 48, or more degrees Celsius and may be maintained for any suitable amount of time (e.g., 10, 15, 30, 45, 60, or more seconds). The drive currents that achieve various target temperatures at the light source 16 may vary with the type of light source 16 utilized.

Accordingly, in some embodiments, the sensor 12 may include an element for sensing a temperature of the light source 16 and/or to sense a temperature of the patient's skin at or adjacent to an interface between the light source 16 and the patient's skin. For example, the temperature sensor may be included within or coupled to the sensor 12 or may be utilized in conjunction with the sensor 12. In certain embodiments, the temperature may be estimated based on physical properties of the light source 16 or detector 18, based on a resistance or by tracking a voltage required to achieve a certain current.

In some embodiments, the system 10 may drive the light source 16 with sufficient power to achieve a predetermined temperature (e.g., approximately 41, 42, 43, 44, 45, 46, or more degrees Celsius) for a predetermined period of time (e.g., 10, 15, 30, 45, 60, or more seconds). Through such techniques, the system 10 may drive the light source 16 to achieve a relatively high SNR that facilitates reliable determination of the patient's pulse rate and selection of appropriate filters based on the pulse rate, as disclosed herein. By way of example, driving the light source 16 with a drive current to achieve a predetermined temperature greater than approximately 41 degrees Celsius may enable the system 10 to determine the patient's pulse rate even in relatively noisy environments, where lower drive currents may not be adequate for reliably determining the patient's rate. In some embodiments, the light source 16 may be driven to achieve a relatively high predetermined temperature (e.g., 42, 43, 44, or more degrees Celsius) in order to provide a signal with a high SNR and to facilitate determination of the patient's pulse rate within a relatively short period of time (e.g., less than about 5, 10, 15, 20, 25, or 30 seconds).

The default power level may be desirable at the beginning of a monitoring session or when the sensor 12 and/or the monitor 14 are first powered on, for example. The signal generated by the detector 18 during operation at the default power level may be transmitted from the detector 18 to the pulse rate detection block 80. In some embodiments, the signal may be filtered (e.g., by filter 54 of FIG. 1) to a relatively wide bandwidth (e.g., suitable for human heart rates up to 300 beats per minute) prior to being transmitted to the pulse rate detection block 80.

In certain embodiments, the pulse rate detection block 80 may be configured to process the signal and to determine the patient's heart rate using one or more algorithms. The patient's heart rate may be determined via any suitable technique. For example, one technique for determining a heart rate from the optical signal received from the detector 18 is to count zero crossings. In some embodiments sequences of crests and troughs in the data are identified and used to determine the heart rate. The pulse rate detection block 80 may determine a power spectrum of one or more of the wavelengths and use the power spectrum data to determine and/or to find the heart rate. In certain embodiments, the pulse detection block 80 may determine a fundamental frequency (e.g., the patient's heart rate) of the received signal (e.g., the sampled waveform) based on the frequency at which the signal has a maximum amplitude. Various techniques for determining pulse rate based on the optical signal are described, for example, in U.S. Pat. No. 731,753 entitled “Pulse Oximeter with Parallel Saturation Calculation Modules,” which is incorporated by reference in its entirety for all purposes.

Regardless of the manner in which the patient's pulse rate is determined, the pulse rate may be provided as an input to the filter selection block 82. The filter selection block 82 may be firmware or data processing circuitry of the monitor 14 and may be generally configured to generate, select, and/or change (e.g., adapt) at least one filter 54 used for processing the signal based at least in part on the patient's heart rate. For example, in certain embodiments, the filter selection block 82 may apply a suitable low pass filter to remove noise higher than the patient's heart rate (or related harmonics). Additionally or alternatively, the filter selection block 82 may apply a suitable high pass or band pass filter to remove noise below the patient's heart rate. In some embodiments, the filter selection block 82 may apply multiple band pass filters and/or may apply various combinations of filters (e.g., a comb filter in combination with a low pass filter). Through such filtering techniques, noise at frequencies other than the heart rate may be reduced, and the system may decrease the light source power while maintaining an adequate SNR for patient monitoring.

A suitable filter 54 may be generated and/or selected in any of a variety of manners. Prior to or during the monitoring session, the filter selection block 82 may change at least one filter 54 and/or adjust coefficients of at least one filter 54 based at least in part on the patient's heart rate. In some embodiments, the filter coefficients may be selected from a plurality of discrete coefficients (e.g., preset or stored values within a memory of the monitor 14). Additionally, the filter 54 and/or the filter coefficients may be periodically (e.g., at predetermined intervals) updated or adjusted during the monitoring session. Additionally, in certain embodiments, the filter 54 and/or the filter coefficients may be continuously tuned based on the patient's heart rate. For example, in some embodiments, the filter 54 and/or the filter coefficients may be adjusted if a change in the patient's heart rate are detected (e.g., when the patient's heart rate changes by more than a threshold amount or percentage) during the monitoring session. In certain embodiments, the filter 54 and/or the filter coefficients may be changed if the monitor 14 detects that the filter 54 is not suitably tracking the patient's heart rate (e.g., the filter 54 does not pass frequencies at the patient's heart rate or related harmonics and/or does not block noise at frequencies above or below the patient's heart rate). The monitor 14 may be configured to determine whether the filter 54 is tracking the patient's heart rate based on any of a variety of signal characteristics or metrics, as described in more detail below.

In certain embodiments, the filter selection block 83 may be configured to design and/or generate the filter 54 during the monitoring session (e.g., at runtime) using any suitable filter design algorithm. The filter design algorithm may be configured to design the filter 54 based at least in part on the patient's pulse rate or pulse rate data (e.g., historical pulse data over 1, 2, 3, or more minutes), as determined and/or received at the pulse rate detection block 80, for example.

In other embodiments, the filter selection block 82 may select an appropriate filter 54 from a filter bank, which may include a plurality of filters 54 stored within the monitor 14, the sensor 12, and/or on an external network or device. In certain embodiments, the filter bank may include one or more low pass, high pass, band pass filters, comb filters, and/or any other type of filter suitable for filtering signals based on various patient heart rates. For example, the bank may include one or more low pass filters configured to pass frequencies below about 5, 4.5, 4, 3.5, 3, 2.5, 2, 1.5, or 1 Hz and/or high pass filters configured to pass frequencies greater than about 0.5, 1, 1.5, 2, 2.5, 3, 3.5, or 4 Hz. In some embodiments, the bank may include one or more band pass filters having any suitable pass bandwidth, such as about 0.5, 1, 1.5, 2 or more Hz. The band pass filters may have any suitable frequency bands, such as about 0.5 to 1, 1 to 1.5, 1.5 to 2 Hz. As set forth above, the filter selection block 82 may access the filter bank and/or select an appropriate filter 54 from the filter bank based at least in part on the patient's heart rate. For example, the filter selection block 82 may select and/or apply a bandpass filter having a center frequency that is closest to the patient's fundamental frequency (e.g., the frequency band may be centered about the patient's fundamental frequency). In certain embodiments, the filter selection block 82 may generate a bandpass filter by shifting a prototype filter (e.g., a filter stored in the filter bank) to a desired center frequency, such as by heterodyning the prototype filter to the desired pulse rate using a complex exponential, for example.

In certain embodiments, the filter selection block 82 may select and/or apply a bandpass filter having pass bandwidth sufficient (e.g., broad or wide enough) to accommodate small changes in heart rate such as changes due to a respiratory sinus arrhythmia or other slightly irregular rhythms, for example. In certain embodiments, it may be desirable to select and/or apply the filter 54 to pass frequencies other than the heart rate. For example, some patients may have an atrioventricular block (e.g., a 2^{nd} or 3^{rd} degree AV block) that results in irregular pulses (e.g., a pulse rate with known, predictable, or expected irregularities in the pulse rate) and/or certain patients may experience frequent ectopic beats (e.g., frequencies higher than the pulse rate). In some such cases, the monitor 14 and/or the pulse rate detection block 80 may employ a clustering algorithm to cluster the measured or detected pulses, for example. Through such techniques, the filter selection block 82 and/or the filter selection algorithm may select and/or generate the filter 54 based on the clusters determined by the clustering algorithm.

Similarly, in addition to generating and/or selecting the filter 54 to reduce noise at frequencies other than the patient's heart rate, the filter selection block 82 may generate and/or select one or more filters 54 having certain characteristics (e.g., pass bandwidth) tuned for the particular patient. Thus, in certain embodiments, the pass bandwidth may be selected to accommodate changes (e.g., expected changes) in the patient's heart rate. In some embodiments, the pass bandwidth may be selected based on historical or previous data. For example, the monitor 14 may determine or receive an input indicating that the patient's heart rate has been or is currently changing significantly (e.g., by about 3%, 5%, 10% or more over a period of time, such as 0.5, 1, 2, or more minutes). In such cases, the filter selection block 82 may select and/or modify the filter 54 to have an adequately wide pass bandwidth to accommodate changes in the patient's heart rate. For example, the filter selection block 82 may select the pass bandwidth such that 1, 2, 3, or more standard deviations of past pulses (e.g., historical data or pulses or subset of pulses obtained in prior 1, 2, 3, or more minutes) are accommodated.

In certain embodiments, the filter selection block 82 may generate, access, and/or select a comb filter configured to pass energy at a specific frequency and all harmonics thereof. In a plethysmography signal, most of the energy is at the patient's heart rate. However, harmonics of the heart rate contain energy that contributes to the pulse shape and features of the signal, such as a dicrotic notch, that are useful for signal processing. Thus, the comb filter may be configured to pass the fundamental frequency as well as higher frequency components related to the pulse shape and dicrotic notch. In some embodiments, gain settings of the amplifier 52 may be adjusted to apply a relatively high gain to a fundamental frequency, while less gain is applied to the higher harmonics.

Using the comb filter, the system 10 may be configured to comb the signal received from the detector 18 so that only the energy at integer multiples of the heart rate passes through the filter. For example, if the patient's heart rate is 60 beats per minute (1 Hz), the filter selection block 82 may generate or select a comb filter configured to pass energy at 1 Hz, 2 Hz, 3 Hz, 4 Hz, etc. and to remove or attenuate energy outside this band. In some embodiments, an adaptive comb filtering (ACF) technique may be utilized. In such cases, the system 10 estimates the fundamental frequency over time and adaptively adjusts the comb filter as the patient's heart rate changes. Additionally, in certain embodiments, the comb filter may be modified or convolved with another filter as appropriate. For example, the comb filter may be modified or convolved with a suitable low pass filter so that higher harmonics are attenuated. Various techniques for generating, adapting, and applying comb filters are described in U.S. Pat. No. 7,336,983, entitled “Pulse Oximeter with Parallel Saturation Calculation Modules,” the entirety of which is incorporated herein by reference in its entirety for all purposes.

By selecting and applying suitable filters based at least in part on the patient's heart rate, the noise acquired by the system 10 at the front end may be reduced. Thus, the light source 16 does not require a high current drive to maintain an acceptable SNR for patient monitoring. Accordingly, in such circumstances, the system 10 may be configured to operate at a low power level (e.g., a low power mode or a reduced power mode) or to enable the low power level in which the light drive circuitry 60 is configured to reduce the power provided to the light source 16 after the filter selection block 82 selects and applies the appropriate filter 54 to the signal.

The light drive circuitry 60 may adjust power to the light source 16 via any suitable technique or combination of techniques. For example, the power to the light source 16 may be reduced by one or more of driving less current through the light source 16 (e.g., by limiting a maximum amount of current available to the light source 16), applying shorter duty cycles, by driving the light source 16 less often, or by adjusting a pattern of illumination. Such techniques may be performed alone or in any suitable combination to reduce the power consumed by the light source 16. In certain embodiments, if the light drive technique uses a sinusoid waveform, the amplitude and/or bias of the sinusoid may be adjusted to reduce the power provided to the light source. In some embodiments, the maximum current provided to the light source 16 after application of the filter 54 and/or during the low power mode may be about 15 to 40 mA, about 20 to 30 mA, or to about 25 mA. In some cases, the power to the light source 16 may be automatically reduced by the system 10 after and/or in response to selection and application of the filter 54 by the filter selection block 82. Furthermore, in certain embodiments, the system 10 may reduce the power to the light source 16 to a predetermined level (e.g., a predetermined level or current that is set at the manufacturing stage or prior to the monitoring session) once the filter 54 is applied. By way of example, the system 10 may automatically reduce the maximum available current to the predetermined level of about 25 mA in response to application of the suitable filter 54.

In certain embodiments, the power consumption of the system 10 may be reduced by additionally or alternatively adjusting a sampling rate (or oversampling ratio) or the number of bits output by the ADC 56. For example, after the filter selection block 82 applies the appropriate filter, the system 10 may then reduce (e.g., automatically reduce) the sampling rate of the ADC 56 and/or the number of bits output by the ADC 56 (e.g., by placing the ADC 56 in a 16-bit mode, 13-bit mode, 9-bit mode, or any suitable mode). In certain embodiments, an ADC hardware averaging setting may be varied based on application of the filter 54, the SNR, or signal quality, for example. In some embodiments, the ADC 56 may be adjusted to operate in a differential mode (e.g., the ADC 56 is configured to measure a voltage difference between two pins) or a singled ended mode (e.g., the ADC 56 is configured to measure the voltage difference between one pin and a ground) based on application of the filter 54, the SNR, or signal quality, for example.

In some embodiments, as discussed in more detail below with respect to FIG. 3, the system 10 may evaluate the signal (e.g., signal quality, SNR, and/or other suitable signal characteristic) prior to adjusting the power provided to the light source 16 and/or power provided to other components of the system 10, such as the ADC 56. Accordingly, FIG. 3 is a block diagram of another medical monitoring system with reduced power consumption, in accordance with an embodiment. In the illustrated embodiment, the system 10 includes the sensor 12 and the monitor 14 having the pulse rate detection block 82, the filter selection block 84, the light drive circuitry 60, as well as a signal quality determination block 92. As set forth above, in some embodiments, the signal may be transmitted from the detector 18 to the pulse rate detection block 82, which is configured to determine the patient's heart rate. The filter selection block 84 is configured to generate, select, and/or apply one or more filters 54 based at least in part on the heart rate. After the selected filter 54 is applied to the signal, the signal quality determination block 92 may evaluate the filtered signal to determine whether the filter suitably tracks the patient's heart rate such that the system 10 may reduce the power provided to the light source 16. The signal quality determination block 92 may consider any suitable metrics, such as pulse shape metrics, amplitude, SNR, margins of various physiological parameters such as oxygen saturation, pulse rate, respiration rate, or any combination thereof. For example, the signal quality determination block 92 may evaluate the peak amplitude of the signal. In some such cases, if the peak amplitude of the filtered signal exceeds a predetermined threshold value for a predetermined period of time (e.g., 10, 20, 30, 40, 50, 60, or more seconds), then the signal quality determination block 92 may determine that filter 54 suitably tracks the heart rate, and thus, the power to the light source 16 or to the ADC 56 may be reduced. Accordingly, the light drive circuitry 60 may be instructed and/or signaled to reduce the power provided to the light source 16, as set forth above.

As noted above, in some embodiments, the signal quality determination block 92 may additionally or alternatively determine and/or monitor the SNR of the signal. In such cases, the signal quality determination block 92 may determine that the filter 54 adequately reduces the SNR of the signal and/or that the SNR of the signal is above a predetermined threshold value, and thus, the power to the light source 16 may be reduced. For example, if the signal quality determination block 92 determines that the SNR is above the target SNR threshold (e.g., a desired SNR threshold preset at the manufacturing state or prior to the monitoring session), the light drive circuitry 60 may be instructed and/or signaled to reduce the power provided to the light source 16, as set forth above. In some embodiments, the power to the light source 16 may be reduced by transitioning from the high power mode to the low power mode (e.g., decreasing the maximum available current to the light source 16 from about 50 mA to about 25 mA or other suitable value).

However, in certain embodiments, the power level may be adaptively and/or incrementally adjusted over time based on the monitored SNR and/or based on the degree with which the filter 54 suitably tracks the patient's heart rate (e.g., based on various signal metrics), as determined by the signal quality determination block 92, for example. Thus, the power level to the light source 16 may be adaptively and incrementally adjusted to control the signal to the target SNR. In such embodiments, the system 10 may not alternate between the discrete high power mode or the low power mode, but may decrease the power or the maximum current provided to the light source 16 adaptively and/or incrementally in response to changes in the SNR and/or in response to the determination related to the degree with which the selected filter 54 tracks the signal. By way of example, the filter selection block 84 may select and/or apply one or more filters 54 to the signal based at least in part on the heart rate. The signal quality determination block 92 may monitor the SNR of the signal after application of the one or more filters 54, and the power level (e.g., the maximum available current) of the light source 16 may be adjusted based on the monitored SNR and may be adjusted to control the power to achieve the target SNR. Thus, in such cases, the power level may be adjusted along a continuum (e.g., between about 20 mA to about 60 mA) rather than being adjusted from the high current (e.g., about 50 mA) in the high power mode to the low current (e.g., about 25 mA) in the low power mode after application of the one or more filters 54.

In some embodiments, the system 10 may be configured to adaptively adjust the power level to the light source 16 to achieve a certain target SNR, and the target SNR may remain stable during operation of the system 10, regardless of the power that is being provided to the light source 16 (e.g., regardless of the current provided and/or regardless of whether the system 10 is operating in the default power mode or the low power mode). However, in other embodiments, the target SNR may vary based on the power level or power mode. For example, when operating in the high power mode, the system 10 may provide a power level sufficient to achieve a first target SNR. When the first target SNR range is achieved, the system 10 may then reduce the power level (e.g., to the low power mode) and may also be configured to control the SNR to a second target SNR range, different (e.g., higher or lower) than the first. Thus, when operating at in the high power mode, the system 10 attempts to achieve a higher SNR so as to select a suitable filter 54. When operating in the low power mode, the system 10 may be configured to accept a lower SNR to reduce power consumption while still achieving a signal of suitable quality for monitoring the patient's physiological characteristics, for example.

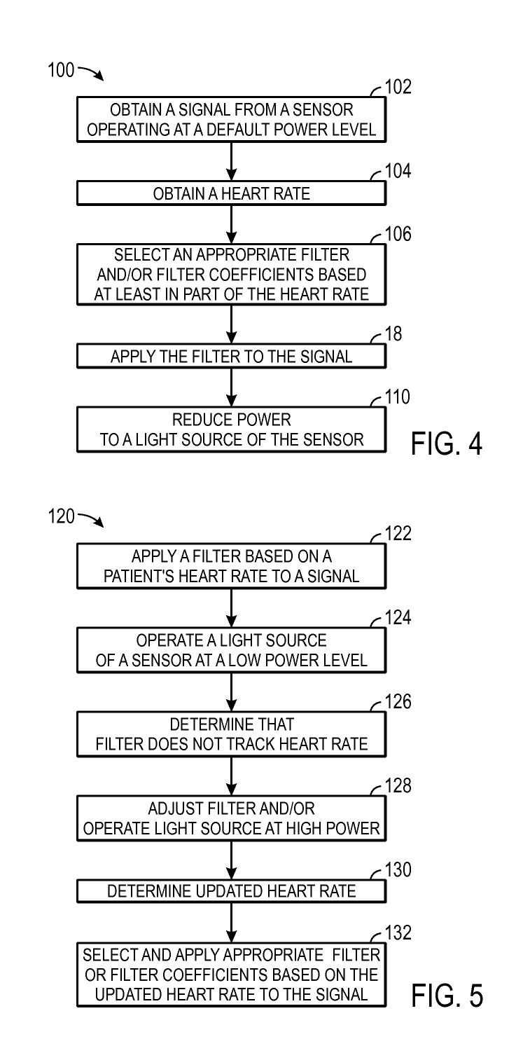

With the foregoing in mind, FIG. 4 and FIG. 5 are flow charts illustrating various methods for operating a monitoring system with reduced power consumption. The methods include various steps represented by blocks. It should be noted that any of the methods provided herein may be performed as an automated procedure by a system, such as system 10. Although the flow charts illustrate the steps in a certain sequence, it should be understood that the steps may be performed in any suitable order and certain steps may be carried out simultaneously, where appropriate. Further, certain steps or portions of the methods may be performed by separate devices. For example, a first portion of the method may be performed by the sensor 12, while a second portion of the method may be performed by the monitor 14. In addition, insofar as steps of the methods disclosed herein are applied to the received signals, it should be understood that the received signals may be raw signals or processed signals. That is, the methods may be applied to an output of the received signals.

With the foregoing in mind, FIG. 4 is a process flow diagram of a method of operating a monitoring system with reduced power consumption, in accordance with an embodiment. The method is generally indicated by number 100. In certain embodiments, the method 100 begins by obtaining a signal, such as a plethysmography signal, at step 102. In some embodiments, the signal may be obtained by a sensing device, such as the sensor 12, and the signal may be provided to a monitoring device, such as the monitor 14. The signal may be obtained while the sensor 12 is operating at the default power level in which the light source 16 is supplied with a first maximum current to achieve a relatively high SNR, for example.

At step 104, a heart rate may be obtained by the monitor 14. The heart rate may be obtained and/or determined by the monitor 14 in any suitable manner. For example, in some embodiments, the monitor 14 may be configured to determine the heart rate based on the signal obtained in step 102. In other embodiments, a signal indicative of the heart rate may be generated by an external device such as an electrocardiogram device or the like, and the monitor 14 may be configured to receive and to process the signal from the external device to determine the patient's heart rate. In yet other embodiments, the heart rate may be provided to the monitor 14 by the external device (e.g., an ECG) or by a user.

At step 106, the monitor 14 may select an appropriate filter 54 and/or filter coefficients based at least in part on the heart rate. As set forth above, in some embodiments, the monitor 14 may access a filter bank stored within the monitor 14, the sensor 12, or an external network or device. The monitor 14 may select one or more suitable filters 54 from the filter bank based at least in part on the patient's heart rate. At step 108, the monitor 14 may apply the one or more selected filters 54 to the signal. As discussed above, applying the one or more filters 54 based on the patient's heart rate to the signal generated by the detector 18 may increase the SNR and enable the power consumed by the system 10 to be reduced (e.g., to enable low power mode). Accordingly, at step 110, the power to the light source 16 of the sensor 12 may be reduced via any suitable techniques. In some embodiments, the power to the light source 16 may be reduced by driving a second maximum current through the light source 16, wherein the second maximum current is lower than the first maximum current (e.g., by limiting a maximum amount of current available to the LED), by applying shorter duty cycles, or by driving the light source 16 less often, or via any other suitable technique or combination of techniques, as discussed above. In some embodiments, the power to the ADC 56 may additionally or alternatively be reduced. As noted above, in some embodiments, the monitor 14 may be configured to monitor the signal quality or any of a variety of suitable signal metrics to determine whether the signal quality and/or the SNR is adequate (e.g., at or above predetermined thresholds) prior to reducing the power to the system 10. Additionally, the power level may be adjusted adaptively or incrementally to maintain a target SNR. In certain embodiments, the power level to the light source 16 may be adjusted from the first maximum current (e.g., about 50 mA) to the second maximum current (e.g., about 25 mA) lower than the first in response to application of the one or more filters 54 and/or in response to adequate signal quality or SNR as determined by the signal quality determination block 92, for example.

In some cases, it may be desirable to adaptively modify a filter as the patient's heart rate changes during a monitoring session. Accordingly, FIG. 5 is a process flow diagram of a method of updating a filter to enable a monitoring system to operate at reduced power, in accordance with an embodiment. Typically the patient's heart rate changes relatively slowly or only changes by a small amount, and thus the adaptive comb filter or the pass band width of the band pass filter may be sufficient to track changes in the patient's heart rate over time. For example, the filter 54 may be configured to accommodate certain changes in the patient's heart rate (e.g., relatively small changes in the patient's heart rate, such as changes of about 1%, 2%, 3%, 4%, 5%, or more). However, in some circumstances, the patient's heart rate may change suddenly (e.g., due to arrhythmia, ectopic beats, or the like) and/or by a relatively large amount or percentage (e.g., by more than about 1%, 2%, 3%, 4%, 5%, or more), and the filter 54 may not track the patient's heart rate in such cases.

With the foregoing in mind, FIG. 5 sets forth a method that is generally indicated by number 120. In certain embodiments, the method 120 begins by applying one or more filters 54 based on the patient's heart rate to a signal, such as a plethysmography signal, in step 122. The signal may be obtained by the sensor 12, and the one or more filters 54 may be applied by the monitor 14, as set forth above. At step 124, the system 10 may reduce the power provided to the light source 16 of the sensor 12 to operate at a low power level (e.g., a low power mode, a reduced power level, a reduced power mode). At step 126, the system 10 may determine that the filter 54 is not tracking the heart rate. The monitor 14 may determine that the filter 54 is not tracking the heart rate based on various metrics, such as decreased SNR, changes in signal metrics or features (e.g., pulse shape metrics, amplitude, etc.), and/or certain changes in measured physiological parameters (e.g., oxygen saturation), for example.

At step 128, the monitor 14 may adjust the filter coefficients, pass bandwidth (e.g., increase the pass bandwidth), the type of filter, or any suitable feature of the one or more filters 54 to accommodate the change in the patient's heart rate. Additionally or alternatively, the monitor 14 may increase the power to the light source 16 (e.g., to the default power level) and/or the ADC 56. The increased power may enable the detector 18 to generate a signal with relatively high SNR for patient monitoring. Additionally, the monitor 14 may process the signal to determine the patient's heart rate (e.g., an updated heart rate), as set forth above with respect to FIG. 2, for example. At step 134, after the heart rate is determined, the monitor 14 may generate or select one or more filters 54 (e.g., updated filters) or filter coefficients based on the patient's updated heart rate. The monitor 14 may apply the one or more updated filters 54 to the signal obtained by the detector 18, and resume monitoring at the low power level as set forth in step 124.

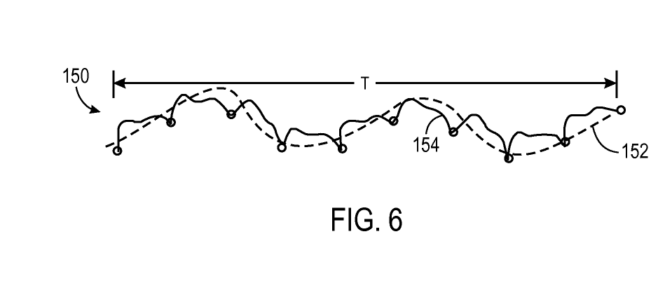

As discussed above, it should be understood that the system 10 may be adapted to additionally or alternatively filter the signal received from the sensor 12 based on the patient's respiration rate. Thus, the system 10 may be configured to determine the patient's respiration rate and to generate, select, and/or apply the filter 54 to the signal received from the sensor 12 based on the patient's respiration rate in order to increase the SNR and to enable the system 10 to operate at reduced power. In such cases, the system 10 may include a respiration rate determination block in lieu or in addition to the pulse rate detection block 80. The respiration rate determination block may be configured to determine the respiration rate via any suitable technique. For example, the respiration rate may be determined via the techniques disclosed in U.S. Publication No. 2011/0071406 entitled “Determining a Characteristic Respiration Rate,” U.S. Publication No. 2011/0021892 entitled “Systems and Methods for Respiration Monitoring,” U.S. Patent Publication No. 2010/0331724 entitled “Determining a Characteristic Blood Pressure, or in U.S. Pat. No. 5,782,756 entitled “Method and Apparatus for in vivo Blood Constituent Analysis,” which are incorporated by reference in their entirety for all purposes. For example, the monitor 14 may be configured to determine the respiration rate based on the signal (e.g., plethysmography signal) received from the sensor 12. As shown in FIG. 6, a plethysmography signal 150 obtained from the patient over a period of time may oscillate (e.g., a baseline of the signal 150 may oscillate in relation to the patient's breathing as indicated by line 152). The signal 150 may include other oscillatory features, such as a pulse 154. The oscillations may be analyzed by the monitor 14 to determine the patient's respiration rate. In some embodiments, the monitor 14 may be configured to transform the signal 150 and to derive the respiration rate based on certain features of the transformed signal 150, such as a ridge corresponding to a characteristic frequency. Additionally, in certain embodiments, the respiration rate may be determined by any suitable external device (e.g., a chest band sensor, a flow meter, or the like), and the respiration rate may be provided (e.g., communicated) to the monitor 14. In certain embodiments, the respiration rate may be input to the monitor 14 by a user.

Regardless of the manner in which the respiration rate is determined, the system 10 may be configured to utilize the respiration rate to generate and/or to select the filter 54 using any of the techniques disclosed above. Thus, the filter 54 may be based on the respiration rate and/or the pulse rate, or one or more different filters 54 may be based on the respiration rate and the pulse rate. The filter 54 based on the respiration rate may be generated and/or selected through any of the techniques set forth above and adapted for use with the respiration rate. In some cases, a typical patient may have a respiration rate of approximately 12-18 breaths per minute, or 0.2-0.3 Hz. Thus, in certain embodiments, the filter 54 may include a low pass filter configured to pass frequencies below about 1, 0.5, 0.4, 0.3, 0.2, or 0.1 Hz, for example, in order reduce baseline shifts due to oscillations due to the patient's breathing Various other types of filters may be selected and/or applied, including those discussed above. Additionally, the filter 54 may be generated and/or selected to accommodate expected respiration rate variability (e.g., based on historical data), as set forth above with respect to pulse rate.

Embodiments of the present disclosure are generally directed toward power efficient medical monitoring systems and methods. To minimize the effects of noise, typically pulse oximetry systems drive the light source with a large amount of current. While the high current drive may minimize the effects of noise and provide a signal having sufficiently high SNR to facilitate patient monitoring, the high current drive also consumes a significant amount of power. The embodiments described herein enable the monitoring system to operate in a power efficient manner in part by generating, selecting, and/or applying suitable filters to the signal at a pre-processing stage (e.g., frontend) to reduce noise in the signal. Such filtering techniques may in turn enable the system to lower the current drive to the light source, while maintaining an adequate SNR for patient monitoring.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that certain elements of the disclosed embodiments may be combined or exchanged with one another.

What is claimed is:

1. A monitor comprising:
a sensor interface configured to obtain a signal from a sensor applied to a patient;
data processing circuitry configured to:
obtain a heart rate of the patient;
select a filter based at least in part on the heart rate; and
filter the signal using the selected filter to reduce noise at frequencies other than the heart rate; and
a light source drive circuitry configured to reduce power to a light source of the sensor after the selected filter is applied to the signal.

2. The monitor of claim 1, wherein the heart rate is determined by the monitor based on the signal.

3. The monitor of claim 1, comprising a memory having a filter bank comprising one or more filters, wherein the data processing circuitry is configured to access the filter bank and to select the filter from the filter bank.

4. The monitor of claim 1, wherein the selected filter comprises a band pass filter.

5. The monitor of claim 4, wherein the monitor is configured increase a pass bandwidth of the band pass filter if the monitor determines that the band pass filter does not track the patient's heart rate.

6. The monitor of claim 1, wherein the selected filter comprises a comb filter.

7. The monitor of claim 6, wherein the monitor is configured to estimate the fundamental frequency over time and to adaptively adjust the comb filter as the heart rate changes.

8. The monitor of claim 1, wherein the light source drive circuitry is configured to increase the power to the light source of the sensor if the monitor determines that the selected filter does not track the heart rate.

9. The monitor of claim 1, wherein the light source drive circuitry is configured to reduce the power to the light source by reducing a maximum current available to the light source of the sensor.

10. A monitoring system comprising:
a sensor interface configured to receive a plethysmography signal from a sensor;
circuitry configured to:
obtain a heart rate of the patient;
select a filter based at least in part on a heart rate of the patient;
apply the filter to the plethysmography signal to increase a signal to noise ratio of the plethysmography signal; and
reduce power to at least one light source of the sensor after the filter is applied to the plethysmography signal.

11. The monitoring system of claim 10, wherein the circuitry is configured to determine the heart rate of the patient based on the plethysmography signal.

12. The monitoring system of claim 10, wherein the circuitry is configured to determine whether the filter tracks the heart rate during a monitoring session.

13. The monitoring system of claim 12, wherein the circuitry is configured to adjust the filter if it is determined that the filter does not track the heart rate.

14. The monitoring system of claim 12, wherein the circuitry is configured to increase the power to the at least one light source of the sensor if it is determined that the filter does not track the heart rate.

15. The monitoring system of claim 10, further comprising the sensor providing the plethysmography signal to the sensor interface.

16. A method for monitoring a patient comprising:
receiving a signal from a sensor applied to a patient;
determining a heart rate of the patient from the signal;
selecting a filter based at least in part on the heart rate of the patient;
applying the filter to the signal; and
reducing power to a light source of the sensor after the filter is applied to the signal.

17. The method of claim 15, comprising increasing the power to the light source if it is determined that the filter does not track the heart rate.

18. The method of claim 15, wherein reducing the power to the light source comprises reducing a maximum current available to the light source.

19. The method of claim 15, wherein the filter is a band pass filter.

20. The method of claim 18, comprising adjusting the pass bandwidth of the filter if it is determined that the filter does not track the heart rate.

21. The method of claim 15, wherein the filter is an adaptive comb filter.
